(19) United States
(12) Patent Application Publication
(10) Pub. No.: US 2025/0222058 A1
(43) Pub. Date: Jul. 10, 2025

(54) DIETARY COMPOSITIONS

(71) Applicant: Melaleuca, Inc., Idaho Falls, ID (US)

(72) Inventors: Jordan Joy, Idaho Falls, ID (US); Nasser A. Fredj, Idaho Falls, ID (US)

(21) Appl. No.: 19/009,392

(22) Filed: Jan. 3, 2025

Related U.S. Application Data
(60) Provisional application No. 63/618,255, filed on Jan. 5, 2024.

(51) Int. Cl. A61K 36/605; A23L 2/66; A23L 33/00; A61K 33/24; A61K 36/48; A61K 47/36; A61K 47/42; A61P 3/04; A61P 3/08

(52) U.S. Cl. CPC A61K 36/605; A23L 2/66; A23L 33/30; A61K 33/24; A61K 36/48; A61K 47/36; A61K 47/42; A61P 3/04; A61P 3/08

(57) ABSTRACT

This document provides dietary compositions as well as methods for using dietary compositions to stabilize glucose levels and/or aid in weight loss. For example, a dietary composition including a bean extract and/or a mulberry extract; chromium (e.g., a chromium oligofructose complex); one or more animal proteins and/or one or more plant proteins; one or more fibers; and a cinnamon extract is provided.

### CROSS-REFERENCE TO RELATED APPLICATIONS

This application claims the benefit of priority to U.S. Provisional Application No. 63/618,255, filed on Jan. 5, 2024, the contents of which is hereby incorporated by reference in its entirety.

### TECHNICAL FIELD

Provided herein are dietary compositions. For example, this document relates to methods and materials useful in supplementing the diet of humans or animals to, for example, help control blood glucose levels and/or aid in weight loss efforts.

### BACKGROUND

Obesity and impaired glucose tolerance are two independent, but interrelated, diseases with very high prevalence in first world countries. Nearly 70% of Americans are overweight or obese. Similarly, almost 50% of Americans have diabetes or prediabetes.

The population of people considered “overweight” has reached epidemic levels, which not only increases the risk of disease in those individuals, but also puts a strain on the healthcare industry as a whole. In fact, obesity is considered by some health authorities to be a leading cause of disease throughout the world, and particularly the United States.

The blood sugar concentration or blood glucose level is the amount of glucose (sugar) present in the blood of a human or animal. Normally in mammals, the body maintains blood glucose levels at a reference range between about 3.6 and 5.8 mM (mmol/L, i.e., millimoles/liter). When healthy, the body's homeostatic mechanisms keep blood glucose levels within a narrow range. Blood sugar levels can vary over the course of the day and spike after a meal, particularly after a sugar-rich meal. Moreover, certain medical conditions, such as diabetes, can interrupt the body's homeostatic mechanisms that maintain blood glucose levels within a narrow range, and thus blood glucose levels may spike even further after a meal for a diabetic. Severe stress, such as trauma, stroke, myocardial infarction, surgery, illness, or poor sleep can also impact a body's hemostatic mechanisms. High blood sugar levels can have a variety of adverse health effects, including weight gain, kidney damage, neurological damage, cardiovascular damage, and retina damage.

Dietary interventions, including dietary compositions, have been shown to have some efficacy for controlling blood glucose levels or losing weight. A wholistic solution targeting both offers a comprehensive solution and is desirable.

### SUMMARY

Provided herein are dietary compositions that can be consumed to help control blood glucose concentration and/or aid in weight loss in a mammal. In some cases, a dietary composition provided herein can work in conjunction with foods being consumed. For example, a dietary composition provided herein can improve insulin response to a meal in a subject. In some embodiments, a dietary composition provided herein can increase health by increasing satiety in a mammal.

Dietary compositions provided herein can include: a bean extract and/or a mulberry extract; a dairy protein and/or a plant protein; a fiber; a chromium; and a cinnamon extract.

In some embodiments, the dietary composition further comprises milk thistle.

In some embodiments, the dietary composition comprises about 25 mg to about 500 mg of the bean extract per serving. In some embodiments, the dietary composition comprises about 50 mg to about 200 mg of the bean extract per serving.

In some embodiments, the bean extract is a Phaseolus vulgaris extract.

In some embodiments, the dietary composition comprises about 5 g to about 40 g of the dairy protein, plant protein, or a combination thereof per serving. In some embodiments, the dietary composition comprises about 10 g to about 20 g of the dairy protein, plant protein, or a combination thereof per serving.

In some embodiments, the dietary composition comprises a dairy protein selected from whey protein isolate, whey protein concentrate, a casein, a caseinate, and a combination thereof.

In some embodiments, the dietary composition comprises about 3 g to about 25 g of the fiber per serving. In some embodiments, the dietary composition comprises about 5 g to about 10 g of the fiber per serving.

In some embodiments, the fiber comprises beta-glucan, inulin, soluble corn fiber, or a combination thereof. In some embodiments, the fiber comprises beta-glucan, inulin, and soluble corn fiber.

In some embodiments, the dietary composition comprises about 17 μg to about 600 μg of elemental chromium per serving. In some embodiments, the dietary composition comprises about 35 μg to about 200 μg of elemental chromium per serving.

In some embodiments, the chromium is present as a chromium oligofructose complex.

In some embodiments, the dietary composition comprises about 40 mg to about 200 mg of a cinnamon extract per serving. In some embodiments, the dietary composition comprises about 100 mg to about 140 mg of a cinnamon extract per serving.

In some embodiments, the cinnamon extract is a 25:1 extract.

In some embodiments, the bean extract is a Phaseolus vulgaris extract; the dairy protein is a combination of whey protein isolate, whey protein concentrate, and calcium caseinate; the fiber is a combination of inulin, beta-glucan, and soluble corn fiber; the chromium is present as a chromium oligofructose complex.

In some embodiments, the dietary composition is incorporated as part of a powdered shake mix.

In some embodiments, the dietary composition stabilizes blood glucose concentration in a mammal. In some embodiments, the dietary composition slows the rate of glucose absorption in a mammal. In some embodiments, the dietary composition increases satiety in a mammal. In some embodiments, the dietary composition decreases hunger cravings in a mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features and advantages will be apparent from the description and drawings, and from the claims.

### DESCRIPTION OF DRAWINGS

FIG. 1A is a plot showing pre-study and post-study waist circumference. −3.5 cm, −3.1%, ES=0.32, p=0.056, 1-β=0.50.

FIG. 1B is a plot showing pre-study and post-study handgrip strength. +1.3 kg, +2.5%, ES=0.13, p=0.257, 1-β=0.19

FIG. 1C is a plot showing pre-study and post-study weight. −1.7 lbs, −0.9%, ES=0.05, p=0.088, 1-β=0.40.

FIG. 2A is a plot showing pre-study and post-study fasting glucose. −1.2 mg/dL, −1.1%, ES=0.13, p=0.62, 1-β=0.08.

FIG. 2B is a plot showing pre-study and post-study 30 min glucose. −12.6 mg/dL, −7.6%, ES=0.71, p=0.01, 1-β=0.75.

FIG. 2C is a plot showing pre-study and post-study 60 min glucose. −2.83 mg/dL, −2.4%, ES=0.10, p=0.55, 1-β=0.09.

FIG. 2D is a plot showing pre-study and post-study 120 min glucose. −6.0 mg/dL, −5.5%, ES=0.42, p=0.045, 1-β=0.54.

FIG. 2E is a plot showing pre-study and post-study glucose AUC. −702.5a.u., −4.7%, ES=0.38, p=0.041, 1-β=0.56.

FIG. 3A is a plot showing pre-study and post-study HbA1C. −0.24%, −4.3%, ES=0.58, p<0.001, 1-β=0.99.

FIG. 3B is a plot showing pre-study and post-study insulin. +0.85 uIU/mL, +4.4%, ES=0.12, p=0.48, 1-β=0.10.

FIG. 3C is a plot showing pre-study and post-study HOMA-IR. +0.3, +3.4%, ES=0.10, p=0.43, 1-β=0.11.

FIG. 4A is a plot showing pre-study and post-study total cholesterol. −6.9 mg/dL, −3.5%, ES=0.21, p=0.23, 1-β=0.2

FIG. 4B is a plot showing pre-study and post-study total: HDL. −0.41, −8.6%, ES=0.28, p=0.045, 1-β=0.54

FIG. 4C is a plot showing pre-study and post-study HDL. +2.6 mg/dL, +6.4%, ES=0.27, p=0.07, 1-β=0.45

FIG. 4D is a plot showing pre-study and post-study LDL. −4.7 mg/dL, −4.1%, ES-0.17, p=0.38, 1-β=0.13.

FIG. 4E is a plot showing pre-study and post-study VLDL. −4.8 mg/dL, −9.6%, ES=0.35, p=0.10, 1-β=0.37

FIG. 4F is a plot showing pre-study and post-study triglycerides. −24.1 mg/dL, −9.3%, ES=0.35, p=0.11, 1-β=0.36.

FIG. 5A is a plot showing between-week post-OGTT energy. +15 mm, +34.2%, ES=1.02, p=0.029, 1-β=0.63.

FIG. 5B is a plot showing between-week post-OGTT fatigue. −1.8 mm, −5.6%, ES=0.10, p=0.74, 1-β=0.06.

FIG. 6A is a plot showing between-week post-OGTT appetite. W0 Within-Day: +19.5 mm, +48.5%, ES=0.84, p=0.001, 1-β=0.99. W12 Within-Day: +5.8 mm, +13.6%, ES=0.25, p=0.385, 1-β=0.13. −10.9 mm, −18.3%, ES=0.46, p=0.21, 1-β=0.23.

FIG. 6B is a plot showing between-week post-OGTT fullness. +12.7 mm, +44.4%, ES=0.60, p=0.018, 1-β=0.72; * p<0.05.

FIG. 6C is a plot showing between-week post-OGTT warmness. W0 Within-Day: −18.9 mm, −38.4%, ES=1.20, p=0.009, 1-β=0.82. Between-week, post-OGTT: +22.1 mm, +44.8%, ES=1.08, p=0.011, 1-β=0.79.

FIG. 6D is a plot showing between-week post-OGTT HMCYE. W0 Within-Day: +13.5 mm, +29.9%, ES=0.68, p=0.012, 1-β=0.78. W12 Within-Day: +5.4 mm, +11.2%, ES=0.23, p=0.12, 1-β=0.34. Between-week, post-OGTT: −4.9 mm, −8.4%, ES=0.20, p=0.457, 1-β=0.11.

FIG. 6E is a plot showing between-week post-OGTT hunger. −7.7 mm, −12.6%, ES-0.29, p=0.19, 1-β=0.25.

FIG. 6F is a plot showing between-week post-OGTT desire to eat. W0 Within-Day: +13.1 mm, +29.9%, ES=0.51, p=0.018, 1-β=0.72. W12 Within-Day: +11.1 mm, +28.0%, ES=0.49, p=0.048, 1-β=0.53. Between-week, post-OGTT: −6.2 mm, −10.9%, ES=0.24, p=0.397, 1-β=0.13

FIG. 7A is a plot showing between-week post-OGTT product satisfaction. +21.5 mm, +45.3%, ES=1.12, p=0.002, 1-β=0.96.

FIG. 7B is a plot showing between-week post-OGTT stomach discomfort. −5.6 mm, −18.7%, ES=0.27, p=0.50, 1-β=0.10.

### DETAILED DESCRIPTION

Provided herein are dietary compositions that contain: a bean extract and/or a mulberry extract; one or more animal proteins and/or one or more plant proteins; one or more sources of fiber; one or more sources of chromium; and a cinnamon extract. In some cases, the compositions also can include milk thistle. Such dietary compositions can be consumed to help control blood glucose concentration and/or aid in weight loss in a mammal.

In some cases, the ingredients of dietary compositions provided herein can work in conjunction with foods being consumed. For example, dietary compositions provided herein can improve insulin response to a meal (e.g., when taken with or before a meal consumed by a mammal). In some embodiments, dietary compositions provided herein can stabilize the blood glucose level and/or slow absorption of glucose in a mammal.

In some embodiments, dietary compositions provided herein can increase health by increasing satiety in a mammal. In some embodiments, dietary compositions provided herein can increase satiety by increasing the physical feeling of fullness in the stomach (e.g., over and above that caused by the volume of food consumed) and/or by promoting the release of cholecystokinin (CCK), a hormone that reduces hunger. For example, a mammal consuming dietary compositions provided herein in conjunction with a carrier food source and/or an additional food can perceive feelings of satiety above what would be perceived in the absence of the dietary composition. In some embodiments, dietary compositions provided herein can decrease hunger cravings. In some embodiments, dietary compositions provided herein can support healthy weight loss and/or healthy energy levels.

In some embodiments, dietary compositions provided herein can support a healthy immune system.

### Bean Extract

In some cases, dietary compositions provided herein can include a bean extract. Without wishing to be bound by a particular theory, bean extracts can slow the rate of sugar absorbed from food, reduce the amount of sugar absorbed from food, and/or reduce hunger. For example, a bean extract can include one or more carbohydrate enzyme inhibitors.

In some embodiments, dietary compositions provided herein can include an extract from a seed of a plant from the genus Phaseolus and/or the genus Vigna. Exemplary species of such plants include, without limitation, Phaseolus vulgaris, Phaseolus lunatus, Vigna radiata, and Vigna angularis. In some embodiments, dietary compositions provided herein can include a Phaseolus vulgaris L. extract (e.g., an extract from seed of Phaseolus vulgaris L.)

In some embodiments, a bean extract can have alpha-amylase inhibition of at least about 6% (e.g., via a colorimetric assay measuring the difference in the activity of the alpha-amylase enzyme compared to a pancreatin standard). In some embodiments, a bean extract can have at least about 1,000 units/mg of α-amylase inhibiting activity (e.g., at least about 1,250, about 1,500, about 1,750, or about 2,000 units/mg of α-amylase inhibiting activity). In some embodiments, a bean extract can have about 1,000 to about 2,000 units/mg of α-amylase inhibiting activity. In some embodiments, a bean extract can have about 1,000 to about 1,600 units/mg of α-amylase inhibiting activity.

In some embodiments, a bean extract can contain less than about 30,000 haemagglutinating units (HAU)/g of a phytohaemagglutinin (e.g., less than about 25,000, about 20,000, about 15,000, about 10,000, about 5,000, about 1,000, about 100, about 10, or about 1 HAU/g of a phytohaemagglutinin). In some embodiments, a bean extract can contain about 1 to about 30,000 HAU/g of a phytohaemagglutinin (e.g., about 1 to about 1,000, about 100 to about 10,000, or about 1,000 to about 30,000 HAU/g of a phytohaemagglutinin). In some embodiments, a bean extract can contain about 8,000 to about 30,000 HAU/g of a phytohaemagglutinin. In some embodiments, a bean extract is an Phaseolus vulgaris L. extract containing about 1,000 to 1,600 units/mg of an α-amylase inhibitor and about 8,000 to 30,000 HAU/g of a phytohaemagglutinin. See, e.g., International Publication No. WO 2007/071334.

Any appropriate method can be used to obtain a bean extract. For example, extraction with an aqueous or hydroethanolic medium and precipitation with suitable mixtures of ethanol and water can be used to obtain a bean extract. See, e.g., International Publication No. WO 2007/071334.

A dietary composition provided herein can contain any appropriate amount of a bean extract. In some embodiments, a dietary composition provided herein can contain about 25 mg to about 300 mg of a bean extract per serving. For example, a dietary composition provided herein can contain about 25 mg to about 50 mg, about 25 mg to about 75 mg, about 25 mg to about 100 mg, about 25 mg to about 125 mg, about 25 mg to about 150 mg, about 25 mg to about 175 mg, about 25 mg to about 200 mg, about 25 mg to about 225 mg, about 25 mg to about 250 mg, about 25 mg to about 275 mg, about 50 mg to about 75 mg, about 50 mg to about 100 mg, about 50 mg to about 125 mg, about 50 mg to about 150 mg, about 50 mg to about 175 mg, about 50 mg to about 200 mg, about 50 mg to about 225 mg, about 50 mg to about 250 mg, about 50 mg to about 275 mg, about 50 mg to about 300 mg, about 75 mg to about 100 mg, about 75 mg to about 125 mg, about 75 mg to about 150 mg, about 75 mg to about 175 mg, about 75 mg to about 200 mg, about 75 mg to about 225 mg, about 75 mg to about 250 mg, about 75 mg to about 275 mg, about 75 mg to about 300 mg, about 100 mg to about 125 mg, about 100 mg to about 150 mg, about 100 mg to about 175 mg, about 100 mg to about 200 mg, about 100 mg to about 225 mg, about 100 mg to about 250 mg, about 100 mg to about 275 mg, about 100 mg to about 300 mg, about 125 mg to about 150 mg, about 125 mg to about 175 mg, about 125 mg to about 200 mg, about 125 mg to about 225 mg, about 125 mg to about 250 mg, about 125 mg to about 275 mg, about 125 mg to about 300 mg, about 150 mg to about 175 mg, about 150 mg to about 200 mg, about 150 mg to about 225 mg, about 150 mg to about 250 mg, about 150 mg to about 275 mg, about 150 mg to about 300 mg, about 175 mg to about 200 mg, about 175 mg to about 225 mg, about 175 mg to about 250 mg, about 175 mg to about 275 mg, about 175 mg to about 300 mg, about 200 mg to about 225 mg, about 200 mg to about 250 mg, about 200 mg to about 275 mg, about 200 mg to about 300 mg, about 225 mg to about 250 mg, about 225 mg to about 275 mg, about 225 mg to about 300 mg, about 250 mg to about 275 mg, about 250 mg to about 300 mg, or about 275 mg to about 300 mg of a bean extract per serving. In some embodiments, a dietary composition provided herein can contain about 50 mg to about 200 mg of a bean extract per serving. For example, a dietary composition provided herein can contain about 50 mg, about 75 mg, about 100 mg, about 125 mg, about 150 mg, about 175 mg, or about 200 mg of a bean extract per serving.

In some embodiments, a bean extract can be present in an amount of about 0.08% to about 1% w/w of a dietary composition provided herein. For example, a bean extract can be present in an amount of about 0.08% to about 0.8% w/w, about 0.08% to about 0.6% w/w, about 0.08% to about 0.4% w/w, about 0.08% to about 0.2% w/w, about 0.2% to about 1% w/w, about 0.2% to about 0.8% w/w, about 0.2% to about 0.6% w/w, about 0.2% to about 0.4% w/w, about 0.4% to about 1% w/w, about 0.4% to about 0.8% w/w, about 0.4% to about 0.6% w/w, about 0.6% to about 1% w/w, about 0.6% to about 0.8% w/w, or about 0.8% to about 1% w/w of a dietary composition provided herein. In some embodiments, a bean extract can be present in an amount of about 0.1%, about 0.15%, about 0.2%, about 0.25%, about 0.3%, about 0.35%, or about 0.4% w/w of a dietary composition provided herein.

### Mulberry Extract

In some cases, a dietary composition provided herein can include mulberry extract. In some embodiments, dietary compositions provided herein can include an extract from Mulberry leaf (e.g., Morus alba L. or Morus alba). In some embodiments, dietary compositions provided herein can include a mulberry extract that includes 1-deoxynojirimycin (DNJ).

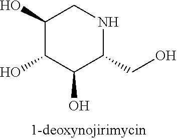 [SMILES: OC[C@H]1NC[C@H](O)[C@@H](O)[C@@H]1O]

In some cases, dietary compositions provided herein can be configured to include a mulberry extract in an amount effective to lower the glycemic index of foods consumed with a dietary composition provided herein. Lowering the glycemic index of foods can reduce the release of insulin, which can result in a more even control of blood glucose levels.

Any appropriate amount of mulberry extract(s) can be included within the dietary compositions provided herein. In some embodiments, dietary compositions provided herein can include about 50 to about 5000 mg of mulberry extract per serving (e.g., about 200 to about 1000 mg, about 400 to about 800 mg, or about 550 to about 650 mg of mulberry extract per serving).

In some embodiments, mulberry extract can be present in an amount of about 0.05% to about 50% w/w of the dietary composition provided herein. For example, about 0.05% to about 40%, about 0.05% to about 30%, about 0.05% to about 20%, about 0.05% to about 10%, about 0.05% to about 1%, about 1% to about 50%, about 1% to about 40%, about 1% to about 30%, about 1% to about 20%, about 1% to about 10%, about 10% to about 50%, about 10% to about 40%, about 10% to about 30%, about 10% to about 20%, about 20% to about 50%, about 20% to about 40%, about 20% to about 30%, about 30% to about 50%, about 30% to about 40%, or about 40% to about 50% w/w of the dietary compositions provided herein.

In some embodiments, a mulberry extract contains 1-deoxynojirimycin present in an amount of about 0.1% to about 99% w/w of the mulberry extract. In some embodiments, a mulberry extract contains 1-deoxynojirimycin present in an amount of about 0.1% to about 10% w/w of the mulberry extract (e.g., about 0.1% to about 8%, about 0.1% to about 6%, about 0.1% to about 4%, about 0.1% to about 2%, about 2% to about 10%, about 2% to about 8%, about 2% to about 6%, about 2% to about 4%, about 4% to about 10%, about 4% to about 8%, about 4% to about 6%, about 6% to about 10%, about 6% to about 8%, or about 8% to about 10% w/w of the mulberry extract.)

### Protein

In some cases, dietary compositions provided herein can include one or more proteins. For example, a dietary composition provided herein can include an animal protein, a plant protein, or a combination thereof. In some embodiments, dietary compositions provided herein can contain about 5 g to about 40 g of protein (e.g., animal protein, plant protein, or a combination thereof) per serving. For example, dietary compositions provided herein can contain about 5 g to about 10 g, about 5 g to about 20 g, about 5 g to about 30 g, about 10 g to about 20 g, about 10 g to about 30 g, about 10 g to about 40 g, about 20 g to about 30 g, about 20 g to about 40 g, or about 30 g to about 40 g of animal protein, plant protein, or a combination thereof per serving. In some embodiments, dietary compositions provided herein contains about 10 g to about 20 g of animal protein, plant protein, or a combination thereof per serving. In some embodiments, a dietary composition provided herein can contain about 5 g, about 10 g, about 15 g, about 20 g, about 25 g, about 30 g, about 35 g, or about 40 g of animal protein, plant protein, or a combination thereof per serving. In some cases, one or more proteins included in a dietary composition provided herein can be hydrolyzed.

In some embodiments, each protein (e.g., each animal protein and/or plant protein) in the dietary compositions provided herein can independently be present in an amount of about 5% to about 10%, about 5% to about 20%, about 5% to about 30%, about 5% to about 40%, about 5% to about 50%, about 5% to about 60%, about 5% to about 70%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80% w/w of the dietary composition.

In some embodiments, dietary compositions provided herein can contain one or more animal proteins (e.g., protein from an animal source). Non-limiting examples of an animal protein include a dairy protein, beef isolate, chicken isolate, collagen, and salmon protein.

Dietary compositions provided herein can contain any amount of an animal protein. For example, dietary compositions provided herein can contain from about 1 g to about 40 g of an animal protein per serving (e.g., about 1 g to about 10 g, about 1 g to about 20 g, about 1 g to about 30 g, about 5 g to about 10 g, about 5 g to about 20 g, about 5 g to about 30 g, about 5 g to about 40 g, about 10 g to about 20 g, about 10 g to about 30 g, about 10 g to about 40 g, about 20 g to about 30 g, about 20 g to about 40 g, or about 30 g to about 40 g of a dairy protein per serving). In some embodiments, a dietary composition provided herein contains about 1 g, about 2 g, about 5 g, about 10 g, about 11 g, about 12 g, about 13 g, about 14 g, about 15 g, about 20 g, about 25 g, about 30 g, about 35 g, or about 40 g of a dairy protein per serving.

A dietary composition provided herein can contain any number of different types of animal protein. For example, a dietary composition can contain one, two, three, four, five, six, seven, eight, nine, ten, or more different types of animal protein.

In some embodiments, dietary compositions provided herein can contain one or more dairy proteins. Any appropriate dairy protein(s) can be included. Non-limiting examples of dairy proteins include whey, casein, and caseinate. Without wishing to be bound by theory, dairy proteins can help control hunger, increase fat oxidation, and/or reduce subsequent calorie intake. For example, beta-lactoglobulin is a type of dairy protein that can comprise over 50% of the protein in whey and can increase cysteine availability to support glutathione production for healthy antioxidant capacity and/or increase production/formation of various immune cells.

Dietary compositions provided herein can contain any appropriate amount of one or more dairy proteins. For example, a dietary composition provided herein can contain from about 1 g to about 40 g of dairy protein per serving (e.g., about 1 g to about 10 g, about 1 g to about 20 g, about 1 g to about 30 g, about 5 g to about 10 g, about 5 g to about 20 g, about 5 g to about 30 g, about 5 g to about 40 g, about 10 g to about 20 g, about 10 g to about 30 g, about 10 g to about 40 g, about 20 g to about 30 g, about 20 g to about 40 g, or about 30 g to about 40 g of a dairy protein per serving). In some embodiments, a dietary composition provided herein can contain about 1 g, about 2 g, about 5 g, about 10 g, about 11 g, about 12 g, about 13 g, about 14 g, about 15 g, about 20 g, about 25 g, about 30 g, about 35 g, or about 40 g of dairy protein per serving.

In some embodiments, a dairy protein is not present in dietary compositions provided herein.

In some embodiments, dietary compositions provided herein can contain whey protein. Any type of whey protein can be included in dietary compositions provided herein. A whey protein can include, for example, a whey protein isolate (WPI) and/or a whey protein concentrate (WPC). For example, WPI is a highly purified, natural source of whey protein that can be incorporated into dietary compositions provided herein. WPI can contain high levels of glycomacropeptides, biologically active peptides that can stimulate the feeling of fullness. WPI can also include biologically active immunoglobulins and lactoferrins.

Dietary compositions provided herein can contain any number of different types of whey protein. For example, dietary compositions can contain one, two, three, four, five, six, seven, eight, nine, ten, or more different types of whey protein.

Any appropriate method can be used to obtain whey protein. For example, milk can be used to obtain a preparation of whey protein. In some embodiments, whey protein can be concentrated and filtered from milk or other dairy sources. In some embodiments, an isolation process can be performed using fluid milk as a source material to obtain a preparation of WPI. In some embodiments, a cold microfiltration process to contain a full balance of undenatured bioactive whey proteins can be used to obtain a preparation of WPI.

Dietary compositions provided herein can contain any appropriate amount of whey protein. In some embodiments, dietary compositions provided herein can contain about 500 mg to about 20 g of whey protein per serving. For example, dietary compositions provided herein can contain about 500 mg to about 5 g, about 500 mg to about 10 g, about 500 mg to about 15 g, about 1 g to about 5 g, about 1 g to about 10 g, about 1 g to about 15 g, about 1 g to about 20 g, about 5 g to about 10 g, about 5 g to about 15 g, about 5 g to about 20 g, about 10 g to about 15 g, about 10 g to about 20 g, or about 15 g to about 20 g of whey protein per serving. In some embodiments, a dietary composition provided herein can contain about 2 g to about 4 g of whey protein per serving. In some embodiments, a dietary composition provided herein can contain about 1 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, or about 15 g of whey protein per serving.

In some embodiments, whey protein can be present in an amount of about 5% to about 40% w/w of the dietary composition.

Whey protein can be present in a dietary composition provided herein in any form, for example, a solution, a powder, or a soluble powder.

In some embodiments, whey protein is not present in dietary compositions provided herein.

In some embodiments, dietary compositions provided herein can contain casein and/or a caseinate. Any type of casein and/or a caseinate can be included in dietary compositions provided herein. For example, dietary compositions provided herein can contain casein, acid casein, micellar casein, calcium caseinate, sodium caseinate, potassium caseinate, or combinations thereof.

Any appropriate method can be used to obtain casein or a caseinate. For example, milk can be used to obtain a preparation of casein or a caseinate. In some embodiments, casein can be obtained from milk via precipitation by acid or coagulation by rennet. In some embodiments, an acid casein is dissolved in a basic solution to obtain a caseinate.

Dietary compositions provided herein can contain any appropriate amount of a casein and/or a caseinate. In some embodiments, dietary compositions provided herein can contain about 100 mg to about 10 g of a casein and/or a caseinate per serving. For example, dietary compositions provided herein can contain about 100 mg to about 500 mg, about 100 mg to about 1 g, about 100 mg to about 2 g, about 100 mg to about 5 g, about 100 mg to about 10 g, about 500 mg to about 1 g, about 500 mg to about 2 g, about 500 mg to about 5 g, about 500 mg to about 10 g, about 1 g to about 2 g, about 1 g to about 5 g, about 1 g to about 10 g, or about 5 g to about 10 g of a casein and/or a caseinate per serving. In some embodiments, dietary compositions provided herein can contain about 800 mg to about 1.5 g of a casein and/or a caseinate per serving. In some embodiments, dietary compositions provided herein can contain about 100 mg, 200 mg, 400 mg, 500 mg, 600 mg, 700 mg, 800 mg, 900 mg, 1 g, about 1.2 g, about 1.5 g, about 2 g, about 2.5 g, or about 3 g of a casein and/or a caseinate per serving. In some embodiments, dietary compositions provided herein can contain about 860 mg of a casein and/or a caseinate per serving. In some embodiments, dietary compositions provided herein can contain about 1 g of a casein and/or a caseinate per serving.

In some embodiments, a casein and/or a caseinate can be present in an amount of about 0.5% to about 5% w/w of the dietary composition.

In some embodiments, calcium caseinate can be present in an amount of about 2% to about 4% w/w of the dietary composition. In some embodiments, calcium caseinate can be present in an amount of about 3.5% w/w of the dietary composition. In some embodiments, calcium caseinate can be present in an amount of about 2% w/w of the dietary composition.

A casein and/or a caseinate can be present in dietary compositions provided herein in any appropriate form. For example, dietary compositions provided herein can contain casein and/or a caseinate in the form of a solution, a powder, or a soluble powder.

In some embodiments, a casein and/or a caseinate is not present in dietary compositions provided herein.

In some embodiments, dietary compositions provided herein can contain a plant protein. Non-limiting examples of suitable plant proteins include soy protein, pea protein, and rice protein. A plant protein can include a protein concentrate and/or a protein isolate. For example, plant protein in a dietary composition provided herein can include a soy protein concentrate, a soy protein isolate, a pea protein concentrate, a pea protein isolate, a rice protein concentrate, a rice protein isolate, or combinations thereof.

Dietary compositions provided herein can contain any number of different types of plant protein. For example, dietary compositions can contain one, two, three, four, five, six, seven, eight, nine, ten, or more different types of a plant protein. Any appropriate method can be used to obtain a plant protein.

Dietary compositions provided herein can contain any amount of plant protein. For example, dietary compositions provided herein can contain from about 1 g to about 40 g of plant protein per serving (e.g., about 1 g to about 10 g, about 1 g to about 20 g, about 1 g to about 30 g, about 5 g to about 10 g, about 5 g to about 20 g, about 5 g to about 30 g, about 5 g to about 40 g, about 10 g to about 20 g, about 10 g to about 30 g, about 10 g to about 40 g, about 20 g to about 30 g, about 20 g to about 40 g, or about 30 g to about 40 g of a plant protein per serving). In some embodiments, a dietary composition provided herein can contain about 1 g, about 2 g, about 5 g, about 6 g, about 6.5 g, about 7 g, about 7.5 g, about 8 g, about 9 g, about 10 g, about 15 g, about 20 g, about 25 g, about 30 g, about 35 g, or about 40 g of plant protein per serving.

In some embodiments, a plant protein is not present in dietary compositions provided herein.

### Fiber

In some cases, dietary compositions provided herein can include fiber. Non-limiting examples of fiber that can be included in a dietary composition provided herein include inulin, soluble corn fiber, psyllium husk, apple fiber, methylcellulose, and beta-glucan. In some embodiments, a dietary composition provided herein can contain about 3 g to about 25 g of fiber per serving. For example, dietary compositions provided herein can contain about 3 g to about 5 g, about 3 g to about 10 g, about 3 g to about 15 g, about 3 g to about 20 g, about 5 g to about 10 g, about 5 g to about 15 g, about 5 g to about 20 g, about 5 g to about 25 g, about 10 g to about 15 g, about 10 g to about 20 g, about 10 g to about 25 g, or about 20 g to about 25 g of fiber per serving. In some embodiments, dietary compositions provided herein can contain about 5 g to about 10 g of fiber per serving. In some embodiments, dietary compositions provided herein can contain about 3 g, about 5 g, about 6 g, about 7 g, about 8 g, about 9 g, about 10 g, about 15 g, about 20 g, or about 25 g of fiber per serving.

In some embodiments, fiber can be present in dietary compositions provided herein in an amount of about 5% to about 60% w/w of the dietary composition. For example, fiber can be present in an amount of about 5% to about 10%, about 5% to about 20%, about 5% to about 30%, about 5% to about 40%, about 5% to about 50%, about 5% to about 60%, about 10% to about 20%, about 10% to about 30%, about 10% to about 40%, about 10% to about 50%, about 10% to about 60%, about 20% to about 30%, about 20% to about 40%, about 20% to about 50%, about 20% to about 60%, about 30% to about 40%, about 30% to about 50%, about 30% to about 60%, about 40% to about 50%, about 40% to about 60%, or about 50% to about 60% w/w of the dietary composition.

In some embodiments, dietary compositions provided herein can contain inulin. Inulin is a group of polysaccharides produced by many types of plants; inulin can be obtained from a plant or can be or synthetically generated. Without wishing to be bound by a particular theory, inulin can increase fiber content and slowing the stomach emptying process. Any type of inulin can be included in dietary compositions provided herein. Examples of inulin that can be included in dietary compositions provided herein include, without limitation, short, medium, and long chain inulin, as well as instant inulin. Inulin can be present in dietary compositions provided herein in any form (e.g., as a solution, a powder, or a soluble powder).

Any appropriate method can be used to obtain inulin. For example, extraction can be used to obtain a preparation of one or more inulin polysaccharides. In some embodiments, inulin can be extracted from naturally-occurring sources such as plants. In particular, chicory or Jerusalem artichoke plants can be used as a source of inulin. In some embodiments, a water extraction process can be performed using chicory as a source material to obtain a preparation of inulin.

Dietary compositions provided herein can contain any appropriate amount of inulin. In some embodiments, a dietary composition provided herein can contain from about 500 mg to about 5 g of inulin per serving. For example, a dietary composition provided herein can contain about 500 mg to about 1 g, about 500 mg to about 2 g, about 500 mg to about 3 g, about 500 mg to about 4 g, about 500 mg to about 5 g, about 1 g to about 2 g, about 1 g to about 3 g, about 1 g to about 4 g, about 1 g to about 5 g, about 2 g to about 3 g, about 2 g to about 4 g, about 2 g to about 5 g, about 3 g to about 4 g, about 3 g to about 5 g, or about 4 g to about 5 g of inulin per serving. In some embodiments, a dietary composition provided herein can contain about 500 mg, 750 mg, 1000 mg, about 1200 mg, about 1300 mg, about 1400 mg, about 1500 mg, about 1600 mg, about 1700 mg, about 1800 mg, about 1900 mg, about 2 g, about 3 g, about 4 g, or about 5 g of inulin per serving.

In some embodiments, inulin can be present in an amount of about 1% to about 10% w/w of the dietary composition.

In some embodiments, inulin can be present in an amount of about 5% w/w of the dietary composition. In some embodiments, inulin can be present in an amount of about 2.5% of the dietary composition.

In some embodiments, dietary compositions provided herein can contain soluble corn fiber. Dietary compositions provided herein can contain any amount of soluble corn fiber. In some embodiments, dietary compositions provided herein can contain from about 1 g to about 15 g of soluble corn fiber per serving. For example, dietary compositions provided herein can contain about 1 g to about 3 g, about 1 g to about 5 g, about 1 g to about 6 g, about 1 g to about 7 g, about 1 g to about 8 g, about 1 g to about 9 g, about 1 g to about 10 g, about 1 g to about 12 g, about 3 g to about 5 g, about 3 g to about 6 g, about 3 g to about 7 g, about 3 g to about 8 g, about 3 g to about 9 g, about 3 g to about 10 g, about 3 g to about 12 g, about 3 g to about 15 g, about 5 g to about 6 g, about 5 g to about 7 g, about 5 g to about 8 g, about 5 g to about 9 g, about 5 g to about 10 g, about 5 g to about 12 g, about 5 g to about 15 g, about 6 g to about 7 g, about 6 g to about 8 g, about 6 g to about 9 g, about 6 g to about 10 g, about 6 g to about 12 g, about 6 g to about 15 g, about 7 g to about 8 g, about 7 g to about 9 g, about 7 g to about 10 g, about 7 g to about 12 g, about 7 g to about 15 g, about 8 g to about 9 g, about 8 g to about 10 g, about 8 g to about 12 g, about 8 g to about 15 g, about 9 g to about 10 g, about 9 g to about 12 g, about 9 g to about 15 g, about 10 g to about 12 g, about 10 g to about 15 g, or about 12 g to about 15 g of soluble corn fiber per serving. In some embodiments, dietary compositions provided herein can contain about 2 g, about 3 g, about 4 g, about 5 g, about 6 g, about 6.5 g, about 6.75 g, about 7 g, about 7.5 g, about 8 g, about 9 g, or about 10 g of soluble corn fiber per serving.

In some embodiments, soluble corn fiber can be present in an amount of about 1% to about 30% w/w of the dietary composition.

In some embodiments, soluble corn fiber can be present in an amount of about 12.5% w/w of the dietary composition. In some embodiments, soluble corn fiber can be present in an amount of about 18% w/w of the dietary composition. In some embodiments, soluble corn fiber can be present in an amount of about 26% w/w of the dietary composition.

In some embodiments, dietary compositions provided herein can contain beta glucan. Beta-glucan is a natural polysaccharide made of glucose. Without wishing to be bound by a particular theory, beta-glucan can increase the viscosity of stomach contents, which can slow stomach emptying, prolong the absorption of energy from a meal, and decrease the absorption of fat. These effects can exert strong control over insulin release in the body, which can reduce cholesterol production, extend satiety, and benefit heart and glycemic health. Any appropriate type of beta glucan can be included in dietary compositions provided herein. Examples of beta glucan include, without limitation, oat, barley, wheat, sorghum, and other beta glucans. Beta glucan can be in any form, for example, a solution, a powder, or a soluble powder.

Dietary compositions provided herein can contain any number of different beta glucans. For example, dietary compositions can contain one, two, three, four, five, six, seven, eight, nine, ten, or more different beta glucans. Any appropriate method can be used to obtain a beta glucan. In some cases, beta glucan can be extracted from one or more naturally-occurring sources. For example, alkaline extraction, acidic extraction, water extraction, or enzymatic extraction can be used to obtain a preparation of beta glucan.

Dietary compositions provided herein can contain any appropriate amount of beta-glucan. In some embodiments, dietary compositions provided herein can contain about 10 mg to about 2 g of beta-glucan per serving. For example, dietary compositions provided herein can contain about 10 mg to about 100 mg, about 10 mg to about 200 mg, about 10 mg to about 300 mg, about 10 mg to about 400 mg, about 10 mg to about 500 mg, about 10 mg to about 800 mg, about 10 mg to about 1 g, about 10 mg to about 1.5 g, about 50 mg to about 100 mg, about 50 mg to about 200 mg, about 50 mg to about 300 mg, about 50 mg to about 400 mg, about 50 mg to about 500 mg, about 50 mg to about 800 mg, about 50 mg to about 1 g, about 50 mg to about 1.5 g, about 50 mg to about 2 g, about 100 mg to about 200 mg, about 100 mg to about 300 mg, about 100 mg to about 400 mg, about 100 mg to about 500 mg, about 100 mg to about 800 mg, about 100 mg to about 1 g, about 100 mg to about 1.5 g, about 100 mg to about 2 g, about 200 mg to about 300 mg, about 200 mg to about 400 mg, about 200 mg to about 500 mg, about 200 mg to about 800 mg, about 200 mg to about 1 g, about 200 mg to about 1.5 g, about 200 mg to about 2 g, about 300 mg to about 500 mg, about 300 mg to about 1 g, or about 300 mg to about 2 g of a beta-glucan per serving. In some embodiments, dietary compositions provided herein contains about 10 mg, about 50 mg, about 100 mg, about 150 mg, about 200 mg, about 250 mg, about 275 mg, or about 300 mg of beta-glucan per serving.

In some embodiments, beta-glucan can be present in an amount of about 0.1% to about 1% w/w of the dietary composition. In some embodiments, beta-glucan can be present in an amount of about 0.84% w/w of the dietary composition. In some embodiments, beta-glucan can be present in an amount of about 0.42% w/w of the dietary composition.

### Chromium

A dietary composition provided herein can include one or more biologically active forms of chromium (Cr). Without wishing to be bound by a particular theory, chromium can interact with insulin via a molecule known as chromodulin, which can help insulin perform its function at the insulin receptor to increase glucose absorption from the blood stream into peripheral tissues. A biologically active form of chromium (Cr) can participate in glucose metabolism by enhancing the effects of insulin. For example, a dietary composition provided herein can include a biologically active form of chromium such that the composition can increase the ability of an insulin-sensitized cell to uptake glucose. In some cases, the biologically active form of chromium can be a low-molecular-weight chromium-binding substance (LMWCr). For example, a biologically active form of chromium can be a naturally-occurring oligo-peptide. In some embodiments, a dietary composition provided herein can include a chromium oligofructose complex (also referred to as a chromium amino acid polyfructose complex). For example, chromium amino acid polyfructose complex is described in U.S. Patent Application Publication No. 2010/0009901 (see e.g., paragraph 210), which is hereby incorporated by reference. In some embodiments, a dietary composition provided herein can include chromium yeast. In some embodiments, a dietary composition provided herein can include a chromium picolinate. In some embodiments, a dietary composition provided herein can include a chromium chelate.

A dietary composition provided herein can contain any appropriate amount of chromium. In some embodiments, a dietary composition provided herein can contain about 17 μg to about 600 μg of elemental chromium per serving. For example, a dietary composition provided herein can contain about 17 μg to about 50 μg, about 17 μg to about 100 μg, about 17 μg to about 150 μg, about 17 μg to about 200 μg, about 17 μg to about 250 μg, about 17 μg to about 300 μg, about 17 μg to about 350 μg, about 17 μg to about 400 μg, about 17 μg to about 450 μg, about 17 μg to about 500 μg, about 17 μg to about 550 μg, about 20 μg to about 100 μg, about 20 μg to about 150 μg, about 20 μg to about 200 μg, about 20 μg to about 250 μg, about 20 μg to about 300 μg, about 20 μg to about 350 μg, about 20 μg to about 400 μg, about 20 μg to about 450 μg, about 20 μg to about 500 μg, about 20 μg to about 550 μg, about 20 μg to about 600 μg, about 25 μg to about 100 μg, about 25 μg to about 150 μg, about 25 μg to about 200 μg, about 25 μg to about 250 μg, about 25 μg to about 300 μg, about 25 μg to about 350 μg, about 25 μg to about 400 μg, about 25 μg to about 450 μg, about 25 μg to about 500 μg, about 25 μg to about 550 μg, about 25 μg to about 600 μg, about 30 μg to about 100 μg, about 30 μg to about 150 μg, about 30 μg to about 200 μg, about 30 μg to about 250 μg, about 30 μg to about 300 μg, about 30 μg to about 350 μg, about 30 μg to about 400 μg, about 30 μg to about 450 μg, about 30 μg to about 500 μg, about 30 μg to about 550 μg, about 30 μg to about 600 μg, about 35 μg to about 100 μg, about 35 μg to about 150 μg, about 35 μg to about 200 μg, about 35 μg to about 250 μg, about 35 μg to about 300 μg, about 35 μg to about 350 μg, about 35 μg to about 400 μg, about 35 μg to about 450 μg, about 35 μg to about 500 μg, about 35 μg to about 550 μg, about 35 μg to about 600 μg, about 50 μg to about 150 μg, about 50 μg to about 200 μg, about 50 μg to about 250 μg, about 50 μg to about 300 μg, about 50 μg to about 350 μg, about 50 μg to about 400 μg, about 50 μg to about 450 μg, about 50 μg to about 500 μg, about 50 μg to about 550 μg, about 50 μg to about 600 μg, about 100 μg to about 200 μg, about 100 μg to about 300 μg, about 100 μg to about 400 μg, about 100 μg to about 500 μg, about 100 μg to about 600 μg, about 200 μg to about 300 μg, about 200 μg to about 400 μg, about 200 μg to about 500 μg, about 200 μg to about 600 μg, about 300 μg to about 400 μg, about 300 μg to about 500 μg, about 300 μg to about 600 μg, about 400 μg to about 500 μg, about 400 μg to about 600 μg, or about 500 μg to about 600 μg of elemental chromium per serving. In some embodiments, a dietary composition provided herein can contain about 35 μg to about 200 μg of elemental chromium per serving. In some embodiments, a dietary composition provided herein can contain about 17 μg, about 25 μg, about 100 μg, about 125 μg, about 150 μg, about 175 μg, about 200 μg, about 250 μg, about 300 μg, about 400 μg, about 500 μg, or about 600 μg of elemental chromium per serving.

In some embodiments, elemental chromium can be present in an amount of about 0.00002% to about 0.01% w/w of the dietary composition. For example, elemental chromium can be present in an amount of about 0.00002% to about 0.0001%, about 0.00002% to about 0.0005%, about 0.00002% to about 0.001%, about 0.00002% to about 0.005%, about 0.0001% to about 0.0005%, about 0.0001% to about 0.001%, about 0.0001% to about 0.005%, 0.0001% to about 0.01%, about 0.0005% to about 0.001%, about 0.0005% to about 0.005%, about 0.0001% to about 0.01%, about 0.001% to about 0.005%, about 0.001% to about 0.01%, or about 0.005% to about 0.01% w/w of the dietary composition.

In some embodiments, a chromium containing compound (e.g., chromium oligofructose complex or a chromium yeast) can be present in an amount of about 0.01% to about 3% w/w of the dietary composition.

In some embodiments, a chromium yeast can be present in an amount of about 0.01% to about 3% w/w of the dietary composition (e.g., about 0.01% to about 0.05%, about 0.01% to about 0.1%, about 0.01% to about 0.5%, about 0.01% to about 1%, about 0.01% to about 1.5%, about 0.01% to about 2%, about 0.01% to about 2.5%, about 0.05% to about 0.1%, about 0.05% to about 0.5%, about 0.05% to about 1%, about 0.05% to about 1.5%, about 0.05% to about 2%, about 0.05% to about 2.5%, about 0.05% to about 3%, about 0.1% to about 0.5%, about 0.1% to about 1%, about 0.1% to about 1.5%, about 0.1% to about 2%, about 0.1% to about 2.5%, about 0.1% to about 3%, about 0.5% to about 1%, about 0.5% to about 1.5%, about 0.5% to about 2%, about 0.5% to about 2.5%, about 0.5% to about 3%, about 1% to about 1.5%, about 1% to about 2%, about 1% to about 2.5%, about 1% to about 3%, about 1.5% to about 2%, about 1.5% to about 2.5%, about 1.5% to about 3%, about 2% to about 2.5%, about 2% to about 3%, or about 2.5% to about 3%, w/w of the dietary composition. In some embodiments, a chromium yeast can be present in an amount of 0.21% w/w of the dietary composition.

In some embodiments, a chromium oligofructose complex can be present in an amount of about 0.01% to about 0.5% w/w of the dietary composition (e.g., about 0.02% to about 0.05%, about 0.02% to about 0.1%, about 0.02% to about 0.3%, about 0.05% to about 0.1%, about 0.05% to about 0.3%, about 0.05% to about 0.5%, about 0.1% to about 0.3%, about 0.1% to about 0.5%, or about 0.3% to about 0.5% w/w of the dietary composition). In some embodiments, a chromium oligofructose complex can be present in an amount of 0.07% w/w of the dietary composition. In some embodiments, a chromium oligofructose complex can be present in an amount of 0.04% w/w of the dietary composition. In some embodiments, a chromium oligofructose complex can be present in an amount of 0.1% w/w of the dietary composition.

When a dietary composition provided herein is consumed, chromium can be stored within the consumer's body over the course of one or more days. Chromium can be maintained at a consistent ratio within the body. Chromium can be incorporated in protein structures (e.g., enzymes and hormones) for use within the body.

### Cinnamon

In some cases, dietary compositions provided herein can include cinnamon. Without wishing to be bound by a particular theory, cinnamon may enhance insulin sensitivity by improving glucose transporter status. In some embodiments, a dietary composition provided herein can be configured to include cinnamon in an amount effective to increase a body's ability to make glycogen.

Cinnamomum verum is a small evergreen tree, the bark of which is a common culinary spice. Most cinnamon sold in the United States and Canada is derived from C. aromaticum or C. cassia, sometimes called “Chinese cinnamon” to distinguish it from C. verum. In some embodiments, a dietary composition provided herein can include an extract from Cinnamomum verum, Cinnamomum aromaticum, and/or Cinnamomum cassia.

In some embodiments, dietary compositions provided herein can include a cinnamon extract that contains phenols. For example, a cinnamon extract incorporated into a dietary composition provided herein can contain between about 10% to 50% phenols (e.g., between 20% and 40% phenols, between 25% and 35% phenols, between 28% and 32% phenols, or about 30% phenols).

Dietary compositions provided herein can contain any appropriate amount of a cinnamon extract and/or cinnamon powder. In some embodiments, dietary compositions provided herein can contain about 1 g to about 5 g of cinnamon powder per serving (e.g., about 1 g to about 2.5 g, 1 g to about 3.5 g, about 2.5 g to about 3.5 g, about 2.5 g to about 5 g of cinnamon powder per serving). In some embodiments, dietary compositions provided herein can contain an amount of cinnamon extract equivalent to about 1 g to about 5 g of cinnamon plant material per serving (e.g., dietary compositions provided herein can contain an amount of cinnamon extract equivalent to about 1 g to about 2.5 g, 1 g to about 3.5 g, about 2.5 g to about 3.5 g, about 2.5 g to about 5 g of cinnamon powder per serving).

In some embodiments, a cinnamon extract included in dietary compositions provided herein can have an extraction ratio of about 25:1, meaning that each milligram of extract is equivalent to 25 milligrams of the plant in its natural state.

In some embodiments, dietary compositions provided herein can contain about 40 mg to about 200 mg of a 25:1 cinnamon extract. For example, dietary compositions provided herein can contain about 40 mg to about 180 mg, about 40 mg to about 160 mg, about 40 mg to about 140 mg, about 40 mg to about 120 mg, about 40 mg to about 100 mg, about 40 mg to about 80 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 200 mg, about 100 mg to about 180 mg, about 100 mg to about 160 mg, about 100 mg to about 140 mg, about 100 mg to about 120 mg, about 120 mg to about 200 mg, about 120 mg to about 180 mg, about 120 mg to about 160 mg, about 120 mg to about 140 mg, about 140 mg to about 200 mg, about 140 mg to about 180 mg, about 140 mg to about 160 mg, about 160 mg to about 200 mg, or about 160 mg to about 180 mg of a 25:1 cinnamon extract per serving. In some embodiments, dietary compositions provided herein can contain about 80 mg, 100 mg, 120 mg, about 140 mg, or about 160 mg of a 25:1 cinnamon extract. In some embodiments, a cinnamon extract (e.g., a 25:1 cinnamon extract) can be present in an amount of about 0.05% to about 2% w/w of the dietary composition. For example, a cinnamon extract can be present in an amount of about 0.05% to about 0.1%, about 0.05% to about 0.5%, about 0.05% to about 1%, about 0.05% to about 1.5%, about 0.1% to about 0.5%, about 0.1% to about 1%, about 0.1% to about 1.5%, about 0.1% to about 2%, about 0.5% to about 1%, about 0.5% to about 1.5%, about 0.5% to about 2%, about 1% to about 1.5%, about 1% to about 2%, or about 1.5% to about 2% w/w of the dietary composition.

In some embodiments, a cinnamon extract (e.g., a 25:1 cinnamon extract) can be present in an amount of about 0.4% w/w of the dietary composition. In some embodiments, a cinnamon extract (e.g., a 25:1 cinnamon extract) can be present in an amount of about 0.2% w/w of the dietary composition.

### Milk Thistle

In some cases, dietary compositions provided herein can include milk thistle. Dietary compositions provided herein can contain any appropriate amount of milk thistle. In some embodiments, dietary compositions provided herein can contain about 50 mg to about 800 mg of milk thistle per serving. For example, dietary compositions provided herein can contain about 50 mg to about 700 mg, about 50 mg to about 600 mg, about 50 mg to about 500 mg, about 50 mg to about 400 mg, about 50 mg to about 300 mg, about 50 mg to about 200 mg, about 50 mg to about 100 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 200 mg, about 200 mg to about 800 mg, about 200 mg to about 700 mg, about 200 mg to about 600 mg, about 200 mg to about 500 mg, about 200 mg to about 400 mg, about 200 mg to about 300 mg, about 300 mg to about 800 mg, about 300 mg to about 700 mg, about 300 mg to about 600 mg, about 300 mg to about 500 mg, about 300 mg to about 400 mg, about 400 mg to about 800 mg, about 400 mg to about 700 mg, about 400 mg to about 600 mg, about 400 mg to about 500 mg, about 500 mg to about 800 mg, about 500 mg to about 700 mg, about 500 mg to about 600 mg, about 600 mg to about 800 mg, about 600 mg to about 700 mg, or about 700 mg to about 800 mg of milk thistle per serving.

In some embodiments, milk thistle can be present in an amount of about 0.05% to about 8% w/w of the dietary composition. For example, milk thistle can be present in an amount of about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.1%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 1% to about 8%, about 1% to about 7%, about 1% to about 6%, about 1% to about 5%, about 1% to about 4%, about 1% to about 3%, about 1% to about 2%, about 2% to about 8%, about 2% to about 7%, about 2% to about 6%, about 2% to about 5%, about 2% to about 4%, about 2% to about 3%, about 3% to about 8%, about 3% to about 7%, about 3% to about 6%, about 3% to about 5%, about 3% to about 4%, about 4% to about 8%, about 4% to about 7%, about 4% to about 6%, about 4% to about 5%, about 5% to about 8%, about 5% to about 7%, about 5% to about 6%, about 6% to about 8%, about 6% to about 7%, or about 7% to about 8% w/w of the dietary composition.

In some embodiments, a dietary composition provided herein can be a composition that includes the ingredients listed in Tables 1A-1H:

TABLE 1A

| Ingredient | Input amount (mg) |
|---|---|
| Protein (WPI, WPC, calcium caseinate) | 8385-15575 |
| Fiber (soluble corn fiber, inulin, oat fiber w/beta | 6240-11600 |
| glucan) | |
| Bean extract | 70-130 |
| Chromium oligofructose complex | 14-26.5 |
| Cinnamon extract | 84-156 |

TABLE 1B

| Ingredient | Input amount (mg) |
|---|---|
| Whey protein isolate | 5935-11025 |
| Whey protein concentrate | 1750-3250 |
| Calcium caseinate | 700-1300 |
| Soluble corn fiber | 5055-9390 |
| Inulin | 1015-1890 |
| Oat fiber w/beta glucan | 170-320 |
| Bean extract | 70-130 |
| Chromium oligofructose complex | 14-26.5 |
| Cinnamon extract | 84-156 |

TABLE 1C

| Ingredient | Input amount (mg) |
|---|---|
| Protein (pea protein) | 8400-1560 |
| Fiber (soluble corn fiber, inulin, oat fiber w/beta | 5040-9375 |
| glucan) | |
| Bean extract | 70-130 |
| Chromium oligofructose complex | 14-26.5 |
| Cinnamon extract | 84-156 |

TABLE 1D

| Ingredient | Input amount (mg) |
|---|---|
| Pea protein | 8400-1560 |
| Soluble corn fiber | 3855-7165 |
| Inulin | 1015-1890 |
| Oat fiber w/beta glucan | 170-320 |
| Bean extract | 70-130 |
| Chromium oligofructose complex | 14-26.5 |
| Cinnamon extract | 84-156 |

TABLE 1E

| Ingredient | Input amount (mg) |
|---|---|
| Protein (soy protein isolate, WPI, | 8050-12700 |
| WPC, calcium caseinate) | |
| Fiber (oat fiber, inulin, oat | 6660-10120 |
| fiber w/beta glucan) | |
| Mulberry extract | 70-130 |
| Chromium oligofructose complex | 12.8-23.9 |
| Cinnamon extract | 84-156 |

TABLE 1F

| Ingredient | Input amount (mg) |
|---|---|
| Soy protein isolate | 5250-7500 |
| Whey protein isolate | 1400-2600 |
| Whey protein concentrate | 700-1300 |
| Calcium caseinate | 700-1300 |
| Oat fiber | 5250-7500 |
| Inulin | 1240-2300 |
| Oat fiber w/beta glucan | 170-320 |
| Mulberry extract | 70-130 |
| Chromium oligofructose complex | 12.8-23.9 |
| Cinnamon extract | 84-156 |

TABLE 1G

| Ingredient | Input amount (mg) |
|---|---|
| Protein (soy protein isolate, WPI, | 8050-12700 |
| WPC, calcium caseinate) | |
| Fiber (soluble corn fiber, inulin, | 6435-9710 |
| oat fiber w/beta glucan) | |
| Mulberry extract | 70-130 |
| Chromium yeast | 42-78 |
| Cinnamon extract | 84-156 |

TABLE 1H

| Ingredient | Input amount (mg) |
|---|---|
| Soy protein isolate | 5250-7500 |
| Whey protein isolate | 1400-2600 |
| Whey protein concentrate | 700-1300 |
| Calcium caseinate | 700-1300 |
| Soluble corn fiber | 5250-7500 |
| Inulin | 1015-1890 |
| Oat fiber w/beta glucan | 170-320 |
| Mulberry extract | 70-130 |
| Chromium yeast | 42-78 |
| Cinnamon extract | 84-156 |

### Other Ingredients

A dietary composition provided herein can further include one or more additional ingredients. For example, a dietary composition provided herein can further include one or more creamers, flavorants, sweeteners, salts, preservatives, fillers, thickeners, colorants, and/or any other food safe additive(s).

In some cases, a dietary composition provided herein can contain one or more fillers or thickeners selected from the following: xanthan gum, sodium carboxymethylcellulose, hydroxyl containing compounds, dextrin and/or dextrin derivatives, carboxymethyl cellulose, hydroxypropyl cellulose, hydroxyethyl cellulose, hydroxypropyl methyl cellulose, methyl cellulose, konjac, collagen, wheat gluten, carrageenan, alginates, propylene glycol alginate, dextrin, pullulan, curdlan, gellan, locust bean gum, guar gum, tara gum, gum tragacanth, pectin, agar, zein, karaya, gelatin, psyllium seed, chitin, chitosan, gum acacia, polyvinyl pyrrolidone, polyethylene oxide, polyvinyl alcohol, or combinations thereof. In some embodiments, a dietary composition provided herein can contain about 50 mg to about 1200 mg (e.g., about 50 mg to about 200 mg, about 200 mg to about 800 mg, about 200 mg to about 1200 mg) of thickener per serving.

In some embodiments, a dietary composition provided herein can contain about 50 mg to about 1200 mg (e.g., about 50 mg to about 200 mg, about 200 mg to about 800 mg, about 200 mg to about 1200 mg) of sodium carboxymethylcellulose per serving.

In some cases, a dietary composition provided herein can contain one or more creamers. For example, a dietary composition provided herein can contain sunflower creamer (e.g., Jerzee IP3 Sunflower CWS). In some embodiments, a dietary composition provided herein can contain about 0.5 g to about 12 g (e.g., about 0.5 g to about 3 g, about 3 g to about 8 g, about 8 g to about 12 g) of sunflower creamer per serving.

In some cases, a dietary composition provided herein can contain one or more of flavorants. For example, a dietary composition provided herein can include vanilla extract, cocoa powder, artificial vanilla flavorant, artificial chocolate flavorant, cream, dried cream extracts, or combinations thereof. In some embodiments, a dietary composition can have a vanilla, chocolate or vanilla caramel flavor profile. In some embodiments, a dietary composition provided herein can contain about 100 mg to about 5000 mg (e.g., about 100 mg to about 1000 mg, about 1000 mg to about 3000 mg, about 3000 mg to about 5000 mg) of flavorant per serving.

In some cases, a dietary composition provided herein can contain one or more salts. Non-limiting examples of salts that can be included in a dietary composition provided herein include sodium chloride and potassium chloride. In some embodiments, a dietary composition provided herein can contain about 10 mg to about 1000 mg (e.g., about 10 mg to about 200 mg, about 200 mg to about 500 mg, about 500 mg to about 1000 mg) of salt (e.g., sodium chloride) per serving.

In some cases, a dietary composition provided herein can contain a sweetener. Non-limiting examples of sweeteners that can be included in a dietary composition provided herein include sucralose, allulose, and stevia. In some embodiments, a dietary composition provided herein can contain about 10 mg to about 600 mg (e.g., about 10 mg to about 20 mg, about 20 mg to about 100 mg, about 100 mg to about 600 mg) of sucralose and/or stevia per serving.

In some embodiments, a dietary composition provided herein can contain about 500 mg to about 10,000 mg (e.g., about 500 mg to about 2000 mg, about 2000 mg to about 5000 mg, about 5000 mg to about 10,000 mg) of allulose per serving.

In some embodiments, a dietary composition provided herein can be substantially free of artificial colors, artificial flavors, and chemical preservatives.

In some embodiments, a dietary composition provided herein can be substantially free of vitamins or mineral fortification other than chromium. In some embodiments, a dietary composition provided herein can include non-chromium vitamins or mineral fortifications.

In some embodiments, a dietary composition provided herein can be a composition that includes ingredients as shown in Table 2:

TABLE 2

| | Serving Input | |
|---|---|---|
| Ingredient | (mg) | % w/w |
| Whey protein isolate | 8481 | 29.20% |
| Whey protein concentrate | 2500 | 8.60% |
| Calcium caseinate | 1000 | 3.40% |
| Corn fiber | 7222 | 24.90% |
| Inulin | 1452 | 5.00% |
| Oat beta-glucan | 244 | 0.80% |
| Bean extract | 100 | 0.30% |
| Chromium oligofructose | 20 | 0.10% |
| complex | | |
| Cinnamon extract | 120 | 0.40% |
| Creamer(s), gum(s), natural | 7861 | 21-32% |
| flavor(s), salt(s), sweetener(s) | | |

In some embodiments, dietary compositions provided herein can be incorporated as part of a powdered drink mix (e.g., a powdered shake mix). For example, dietary compositions provided herein can be added to a meal replacement shake base. The configuration of a suitable shake base will be apparent to one skilled in the art in view of the disclosure herein. The final shake can have any flavor (e.g., chocolate, vanilla, or strawberry). The final serving size can be, for example, about 30 g to be dispersed into about 8 ounces of milk or water (or other suitable liquid) to form one complete serving. The final serving size can be greater or less than about 30 grams (e.g., about 15 or about 60 grams) and can provide more or less than one complete serving (e.g., half a serving or two servings).

In some embodiments, dietary compositions provided herein can be formulated as a powder. In some embodiments, the powdered dietary composition can be mixed with a liquid to make a beverage. For example, the powdered dietary composition can be provided in a single serving package or in a bulk package.

### Methods

Dietary compositions provided herein can aid in weight loss in a mammal (e.g., a human). Weight loss can be examined using any appropriate method (e.g., by using a weight scale). A human's weight can be continually or intermittently monitored over a period of time, e.g., once a day, once a week, once a month. A weight trend may indicate the effectiveness of a dietary composition in lowering a human's weight or maintaining a healthy weight. In some embodiments, dietary compositions provided herein can increase satiety in a mammal and/or can decrease hunger cravings in a mammal.

Dietary compositions provided herein can stabilize blood glucose concentration in a mammal. In some embodiments, dietary compositions provided herein can slow the rate of glucose absorption in a subject. In some cases, dietary compositions provided herein can increase the ability of insulin-sensitized cells to uptake glucose, reduce fasting blood glucose levels, lower the glycemic index of foods consumed therewith, reduce insulin resistance, improve glucose tolerance, up-regulate glucokinase MRNA expression, activate insulin receptors, increase glycogen synthase activity, and/or stimulate the release of insulin. In some embodiments, dietary compositions provided herein can aid in insulin uptake/release, and support normal metabolism of blood sugar.

In some embodiments, dietary compositions provided herein can be consumed with a meal. For example, dietary compositions provided herein can be consumed within 10 minutes before or after consuming a meal (e.g., within 5 minutes of consuming a meal or between bites of a meal), or while consuming a meal. In some embodiments, dietary compositions provided herein can be consumed within about 30 minutes before consuming a meal. In some embodiments, dietary compositions provided herein can be consumed before 1 meal per day. In some embodiments, dietary compositions provided herein can be consumed before 2 meals per day. In some embodiments, dietary compositions provided herein can be consumed before each meal.

In some embodiments, dietary compositions provided herein can be consumed between meals.

In some embodiments, one serving of a dietary composition provided herein can be consumed per day. In some embodiments, two servings of a dietary composition provided herein can be consumed per day.

### EXAMPLES

### Example 1. Exemplary Dietary Compositions

A dietary composition was produced having the ingredients shown in Table 3.

TABLE 3

| | Serving | |
|---|---|---|
| Ingredient | Input (mg) | % w/w |
| Whey protein isolate | 8481 | 29.2% |
| Whey protein concentrate | 2500 | 8.6% |
| Calcium caseinate | 1000 | 3.5% |
| Corn fiber | 7222 | 25% |
| Inulin | 1452 | 5% |
| Oat beta-glucan | 244 | 0.8% |
| Bean extract | 100 | 0.3% |
| Chromium oligofructose | 20 | 0.1% |
| complex | | |
| Cinnamon extract | 120 | 0.4% |
| Creamer(s), gum(s), natural | 7861 | 27.1% |
| flavor(s), salt(s), sweetener(s) | | |

A dietary composition was produced having the ingredients shown in Table 4.

TABLE 4

| Ingredient | Input Amount (mg) |
|---|---|
| Whey protein isolate | 8480 |
| Whey protein concentrate | 2500 |
| Calcium caseinate | 1000 |
| Soluble corn fiber | 7222 |
| Inulin | 1452 |
| Oat fiber w/beta glucan | 244 |
| Bean extract | 100 |
| Chromium oligofructose | 20.4 |
| complex | |
| Cinnamon extract | 120 |
| Creamer(s), gum(s), natural | 10552 |
| flavor(s), salt(s), sweetener(s) | |

A dietary composition was produced having the ingredients shown in Table 5.

TABLE 5

| Ingredient | Input Amount (mg) |
|---|---|
| Pea protein | 12,000 |
| Soluble corn fiber | 5511 |
| Inulin | 1452 |
| Oat fiber w/beta glucan | 244 |
| Bean extract | 100 |
| Chromium oligofructose | 20.4 |
| complex | |
| Cinnamon extract | 120 |
| Creamer(s), gum(s), natural | 7860 |
| flavor(s), salt(s), sweetener(s) | |

A dietary composition was produced having the ingredients shown in Table 6.

TABLE 6

| Ingredient | Input Amount (mg) |
|---|---|
| Soy protein isolate | 7503 |
| Whey protein isolate | 2000 |
| Whey protein concentrate | 1000 |
| Calcium caseinate | 1000 |
| Oat fiber | 7500 |
| Inulin | 1770 |
| Oat fiber w/beta glucan | 244 |
| Mulberry extract | 100 |
| Chromium oligofructose | 18.4 |
| complex | |
| Cinnamon extract | 120 |
| Creamer(s), gum(s), natural | 7745 |
| flavor(s), salt(s), sweetener(s) | |

A dietary composition was produced having the ingredients shown in Table 7.

TABLE 7

| Ingredient | Input Amount (mg) |
|---|---|
| Soy protein isolate | 7503 |
| Whey protein isolate | 2000 |
| Whey protein concentrate | 1000 |
| Calcium caseinate | 1000 |
| Soluble corn fiber | 7500 |
| Inulin | 1452 |
| Oat fiber w/beta glucan | 244 |
| Mulberry extract | 100 |
| Chromium yeast | 60 |
| Cinnamon extract | 120 |
| Creamer(s), gum(s), natural | 8021 |
| flavor(s), salt(s), sweetener(s) | |

### Example 2. Health Assessment Study

### Methods

Participants (n=12) were, on average, borderline prediabetic, with a baseline fasting glucose of about 100 mg/dL and HbA1C of 5.5% compared to the diagnostic criteria of 100-125 mg/dL and >5.6%, respectively.

The participants consumed the formulation shown in Table 8 b.i.d. for 12 weeks. The formulation was consumed one time before their two largest meals of the day. No changes were made to their diet and exercise habits.

TABLE 8

| Ingredient | Input (mg) |
|---|---|
| Whey protein isolate | 8480.58 |
| Whey protein concentrate | 2500.00 |
| Calcium caseinate | 1000.00 |
| Soluble corn fiber | 7222.22 |
| Inulin | 1452.27 |
| Oat fiber w/beta glucan | 243.90 |
| Mulberry leaf extract | 878.00 |
| Chromium oligo complex | 30.60 |
| Bean extract | 100.00 |
| Cinnamon extract 30% | 200.00 |
| Milk thistle 80% | 255.00 |

Before and after the intervention, participants had an oral glucose tolerance test (OGTT; 50 g sugar), weight, waist circumference, handgrip, and visual analogue scale (VAS) tests completed before determination of fasting insulin, HbA1C, and blood lipids. The formulation was consumed prior to glucose consumption during the week 12 OGTT.

VAS were administered before and after the OGTT during pre- and post-intervention testing. Within-day and between-week data were analyzed.

Data were analyzed using paired t-test, α<0.05. Effect sizes(ES) were calculated as Cohen's d; ESs of 0.2-0.49 are small, 0.5-0.79 are medium, and ≥0.8 are large. A post-hoc power (1-β) of ≥0.80 is adequate.

14 participants completed baseline testing and were enrolled in the study. Two participants did not finish. The demographics of the participants were as follows:
Age: 40.8±10.2 y
Screening fasting glucose: 97.9±5.6 mg/dL
Height: 68.8±3.2 in
Weight: 204.5±32.7 lbs
BMI: 30.3±4.0
Sex: 10M, 2F

### Results

Significant improvements in HbA1C, glucose area under the curve (AUC), total to HDL ratio, subjective energy, and subjective satiety were observed. Results (not statistically significant, p>0.05) indicate participants lost weight (−1.7 lbs) and reduced their waist circumference (−3.5 cm) while handgrip strength improved (+1.3 kg), suggesting favorable changes in body composition (loss of fat mass, retention of muscle mass). Weight and waist circumference approached significance (p<0.10). See Table 9.

TABLE 9

| | Baseline | Week 12 | Reference Range |
|---|---|---|---|
| HbA1C (%) | 5.50 | 5.26* | 3.9-5.6 |
| Fasting Glucose (mg/dL) | 100.5 | 99.3 | 70-100 |
| 12 0min Glucose (mg/dL) | 93.2 | 87.2* | 70-100 |
| Glucose AUC (a.u.) | 14,876.3 | 14,173.8* | n/a |
| Insulin (uIU/mL) | 13.1 | 13.9 | 6.0-27.0 |
| Total Cholesterol | 194.2 | 187.3 | 130-200 |
| (mg/dL) | | | |
| Total: HDL Cholesterol | 4.8 | 4.4* | <4.0 |
| HDL (mg/dL) | 42.8 | 45.3† | 40-60 |
| LDL (mg/dL) | 120.1 | 115.4 | <100 |
| VLDL (mg/dL) | 31.2 | 26.3† | 6-30 |
| Triglycerides (mg/dL) | 156.7 | 132.6 | 30-150 |

*p < 0.05;
†p < 0.10. Paired T test.
Subjective data were collected by 100mm visual analogue scale (VAS).

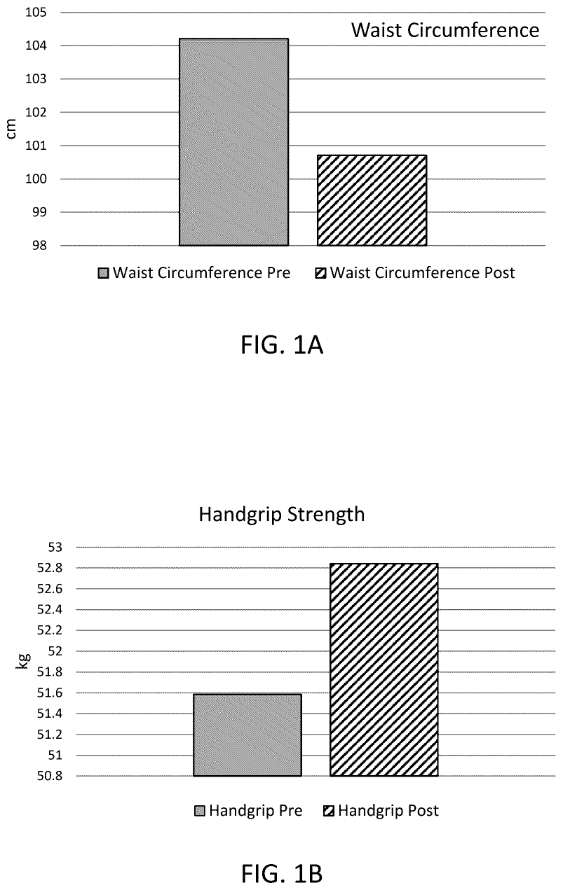

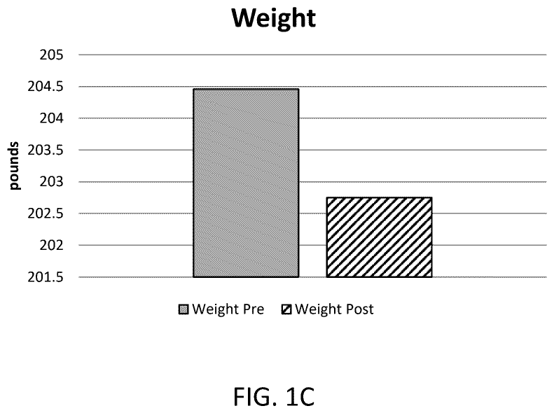

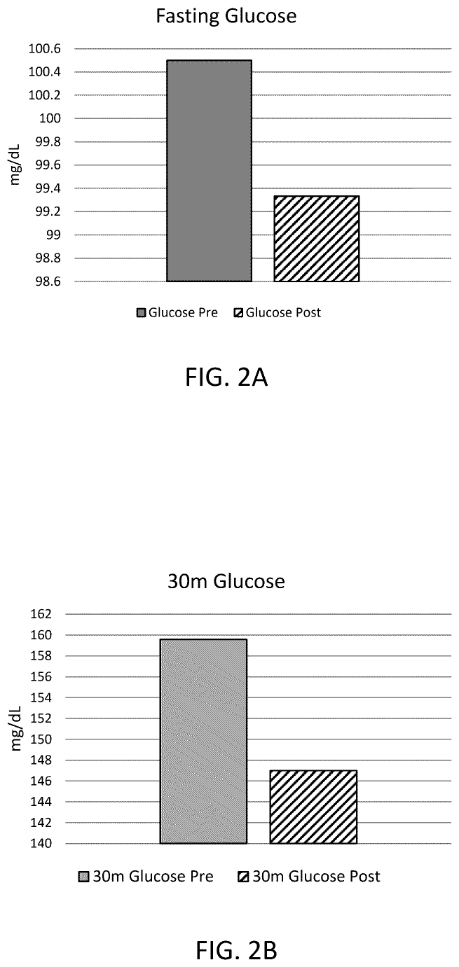

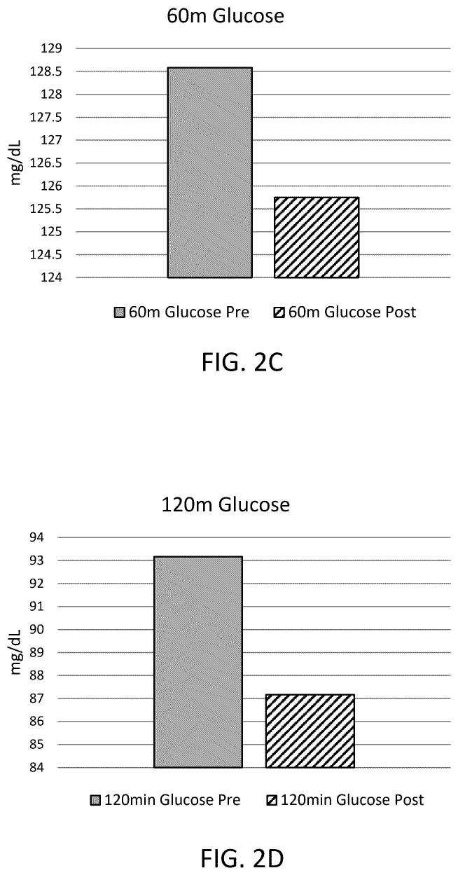

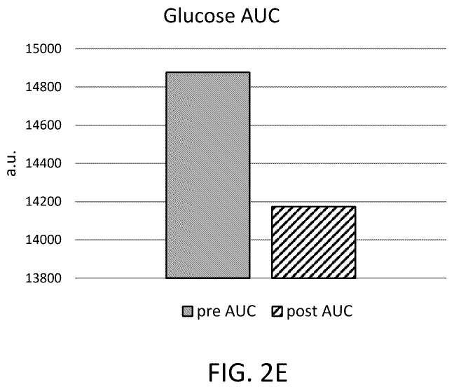

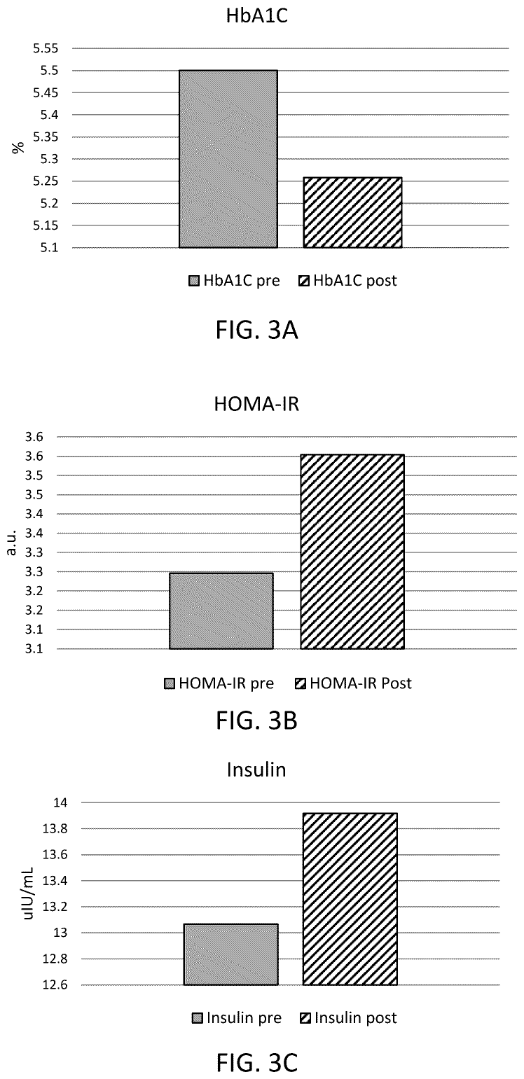

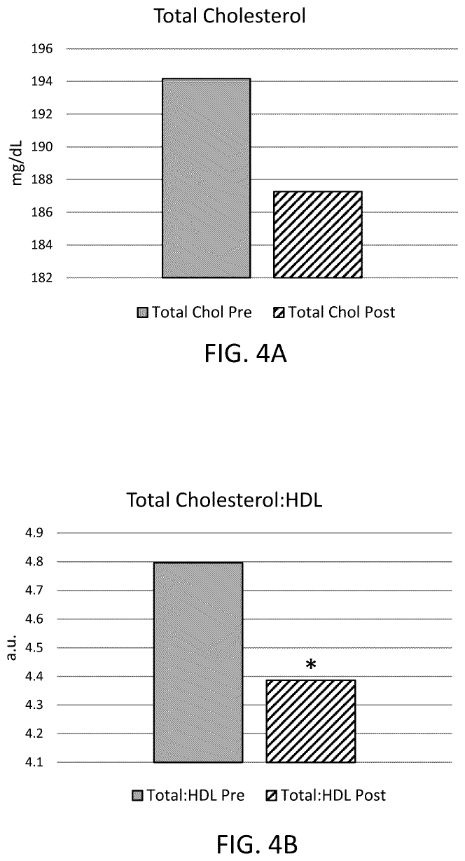

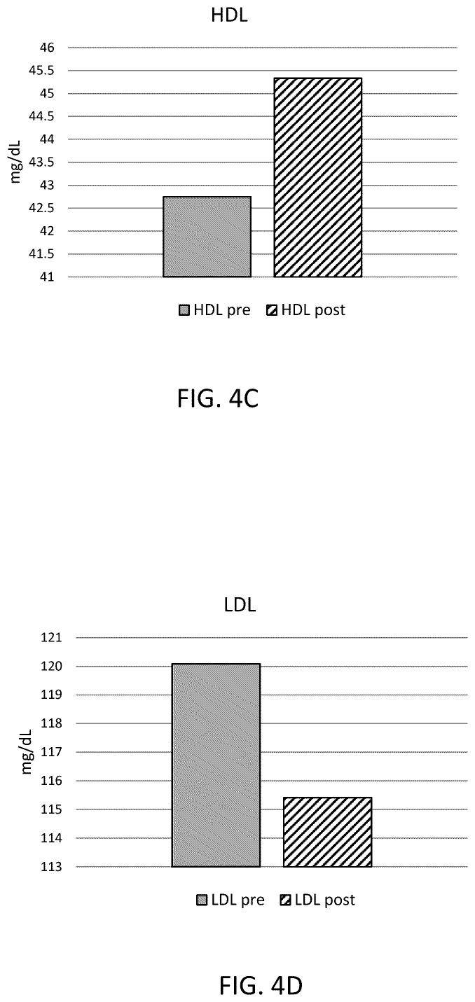

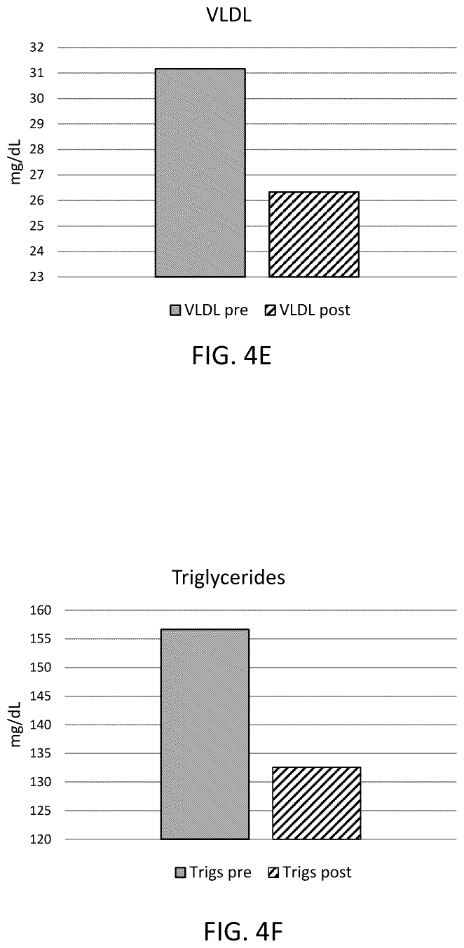

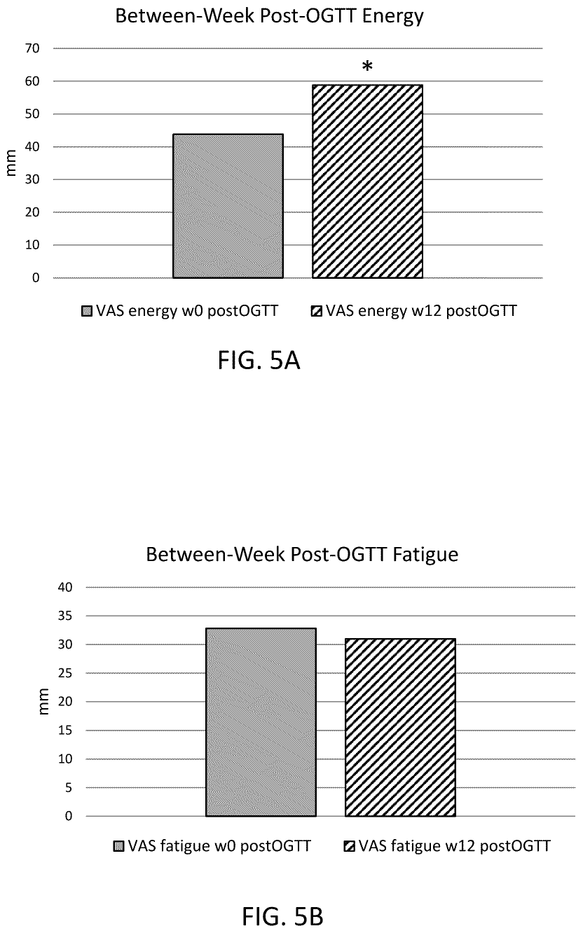

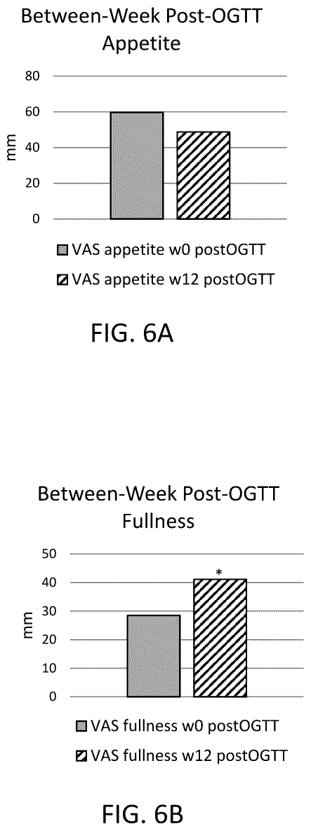

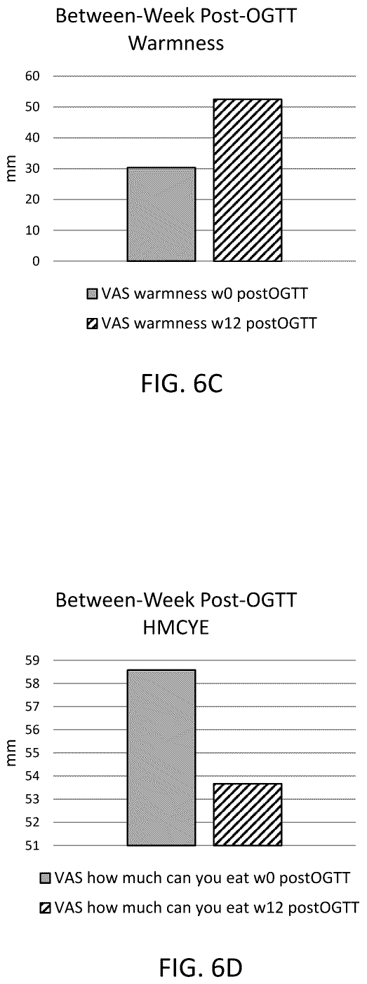

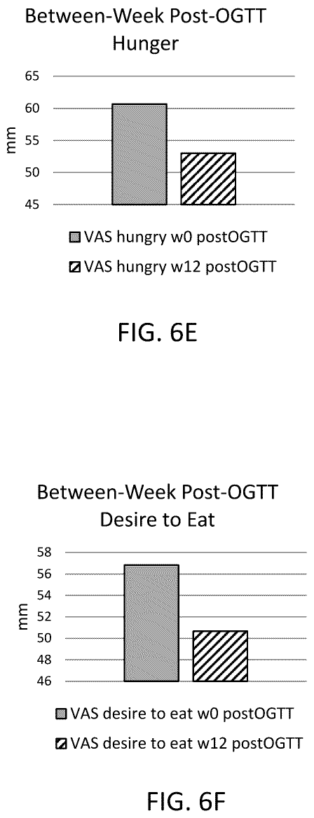

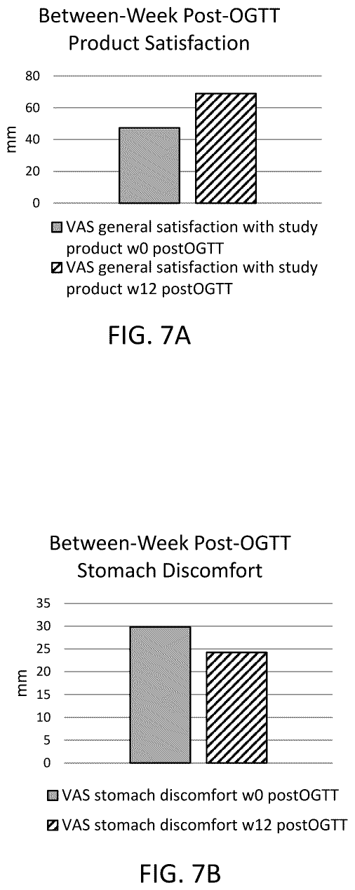

See, also, FIGS. 1A-1C, 2A-2E, 3A-3C, 4A-4F, 5A-5B, 6A-6F, and 7A-7B.

These studies indicated that the administered formulation, containing efficacious doses of protein, fiber, Phaseolus vulgaris, mulberry, milk thistle, cinnamon, and OLIGO® chromium, was effective for improving postprandial blood glucose levels, glycation status, cholesterol profiles, energy levels, and satiety. Study participants did not report any significant adverse effects (SAEs).

### Example 3. Health Assessment Study

### Methods

Another study was designed as a randomized, longitudinal study to assess the acute and 8-week supplementation efficacy of the formulation of Table 10 below and the formulation of Table 4 above.

TABLE 10

| Ingredient | UM/serving |
|---|---|
| Fiber (soluble corn fiber, maltodextrin, | 7 g |
| chicory root fiber) | |
| Protein (soy protein isolate, WPI, | 11 g |
| WPC, calcium caseinate) | |
| Chromium | 120 μg |
| Blend of cinnamon bark extract, ginseng | 1370 mg |
| root extract, mulberry leaf extract, green tea leaf | |
| extract, and sweet potato tuber extract | |

40 (20/20) pre-metabolic male and female subjects to perform the study were recruited. The groups were balanced by gender and BMI. The inclusion criteria were age 40+/−15 years and at least 4 of the following criteria:
BMI 26-32
Total cholesterol >200 mg/dl
HDL <50 mg/dl
Blood pressure sBP >120 mmHg
Fasting glucose >100 mg/dl.

Heart/immunity health status and metabolic, glucose, and lipid profile were checked by clinical and laboratory examination. Energy level and satiety were evaluated using standard visual analog scale (VAS) questionnaires. Volunteers were at least 12 h fasted, received a bread roll with the formulation of Table 10 or the formulation of Table 4, and were allowed to drink water ad libitum.

Primary Efficacy Evaluation: To lower and/or stabilize blood glucose levels in subjects who are prediabetic and increased score on VAS questionnaires for energy levels and satiety. Secondary efficacy Evaluation: improvement in lipid profile.

Subjects received a control in powder form (white canisters or pouches) and received consumer 2 shakes a day (morning and evening) during the duration of the study.

To be included in the study, participants met the following criteria: no intake of vitamins/supplements prior to 4 weeks before inclusion and no medication that would affect endothelial function (e.g., ACE inhibitors, β-blockers), blood glucose (e.g., metformin), or cholesterol level (e.g., statins). Volunteers consented to keep an unchanged lifestyle, level of daily physical activity, and less than 2% weight loss in the last 4 weeks.

Exclusion criteria included generally accepted contraindications to physical exercise; smokers, type 1 and type 2 diabetes; liver and kidney impairments; psychiatric disorders, other disorders of acute or chronic nature (gastrointestinal, pulmonary, renal, cardiac, neurological or psychiatric disorders), known allergies to foods or their ingredients, use of weight-reducing preparations or appetite suppressants, β-blockers, ACE inhibitors, positive COVID-19 PCR test within 12 months, participation in a clinical study within the last 30 days before the beginning of this study or during this study. Health status is checked by clinical and laboratory examination.

Day zero included study information and acceptance; questionnaire-exclusion criteria, health status; complete VAS questionnaire; BMI, blood draw for clinical-, and laboratory parameters; CMA, fasting glucose, and insulin analysis; evaluation of heart and function parameters (sBP, dBP, HR); administration of bread roll and glass of water; and observation of volunteers (blood draw for glucose and insulin analysis).

Day 28 included and Day 56 will include general examination; questionnaire-exclusion criteria, health status; complete VAS questionnaire; blood draw for clinical-, and laboratory parameters (see list below); CMA/eCMA, fasting glucose and insulin analysis; evaluation of heart function parameters (sBP, dBP, HR); administration of formulation Table X as a shake with a bread roll; and volunteer observation, evaluation of aimed parameters (blood draw for postprandial glucose and insulin analysis).

List of parameters and clinical laboratory analysis (according to GCP (Good Clinical Praxis) guidelines):
a. Assessment of oxidative stress level: (extended CMA test)
   i. Total/cellular CMA/eCMA (intra-/extracellular ROS generation)
b. Routine blood test (safety lab):
   i. Haemogram
   ii. Creatinine, urea (kidney function)
   iii. ALT, AST, GGT, bilirubin, ALP (liver function)
c. Metabolic status determination:
   i. Fasting & postprandial glucose (metabolic syndrome evaluation)
   ii. Fasting & postprandial insulin concentration (metabolic syndrome evaluation)
   iii. HbA1c (prediabetes/diabetes evaluation)
   iv. HOMA Index (prediabetes/diabetes evaluation)
d. Lipid profile/Dyslipoproteinemia:
   i. Total cholesterol (evaluation hypercholesterolemia)
   ii. LDL (evaluation of dyslipidemia)
   iii. HDL (evaluation of dyslipidemia)
   iv. VLDL (evaluation of dyslipidemia)

### Results

Interim results at 4 weeks are provided in Table 11.

TABLE 11
Attribute changes after 4 weeks

| | Table 10 | Table 4 |
|---|---|---|
| Attribute | Formulation | Formulation |
| Fasting Glucose | −2.90% | −7.50% |
| Glucose spike @ 1 hr | −11.8% | −16.00% |
| mOGTT Glucose AUC | −6.00% | −10.30% |
| Fasting Insulin | −2.60% | −1.20% |
| mOGTT Insulin AUC | 3.20% | −1.70% |
| HOMA-IR | −4.60% | −9.60% |
| HbA1C | 0.60% | 0.50% |
| Total Cholesterol | 0.90% | −8.60% |
| HDL | 7.40% | 11.70% |
| LDL | −0.80% | −12.60% |
| VLDL | −3.40% | −27.50% |
| Triglycerides | −3.40% | −13.00% |
| Body Weight | −1.00% | −1.00% |
| Fasting ROS | −3.70% | −8.80% |
| mOGTT ROS AUC | −3.20% | −5.30% |
| Fasting mitoROS | −27.70% | −59.60% |
| mOGTT mitoROS AUC | −29.30% | −54.00% |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A dietary composition, comprising:
a bean extract and/or a mulberry extract;
a dairy protein, a plant protein, or a combination thereof;
a fiber;
a chromium; and
a cinnamon extract.

2. The dietary composition of claim 1, wherein the dietary composition further comprises milk thistle.

3. The dietary composition of claim 1 or claim 2, wherein the dietary composition comprises about 25 mg to about 500 mg of the bean extract per serving.

4. The dietary composition of any one of claims 1-3, wherein the dietary composition comprises about 50 mg to about 200 mg of the bean extract per serving.

5. The dietary composition of any one of claims 1-4, wherein the bean extract is a Phaseolus vulgaris extract.

6. The dietary composition of any one of claims 1-5, wherein the dietary composition comprises about 5 g to about 40 g of the dairy protein, plant protein, or a combination thereof per serving.

7. The dietary composition of any one of claims 1-6, wherein the dietary composition comprises about 10 g to about 20 g of the dairy protein, plant protein, or a combination thereof per serving.

8. The dietary composition of any one of claims 1-7, wherein the dietary composition comprises a dairy protein selected from whey protein isolate, whey protein concentrate, a casein, a caseinate, and a combination thereof.

9. The dietary composition of any one of claims 1-8, wherein the dietary composition comprises about 3 g to about 25 g of the fiber per serving.

10. The dietary composition of any one of claims 1-9, wherein the dietary composition comprises about 5 g to about 10 g of the fiber per serving.

11. The dietary composition of any one of claims 1-10, wherein the fiber comprises beta-glucan, inulin, soluble corn fiber, or a combination thereof.

12. The dietary composition of any one of claims 1-11, wherein the fiber comprises beta-glucan, inulin, and soluble corn fiber.

13. The dietary composition of any one of claims 1-12, wherein the dietary composition comprises about 17 μg to about 600 μg of elemental chromium per serving.

14. The dietary composition of any one of claims 1-13, wherein the dietary composition comprises about 35 μg to about 200 μg of elemental chromium per serving.

15. The dietary composition of any one of claims 1-14, wherein the chromium is present as a chromium oligofructose complex.

16. The dietary composition of any one of claims 1-15, wherein the dietary composition comprises about 40 mg to about 200 mg of a cinnamon extract per serving.

17. The dietary composition of any one of claims 1-16, wherein the dietary composition comprises about 100 mg to about 140 mg of a cinnamon extract per serving.

18. The dietary composition of any one of claims 1-17, wherein the cinnamon extract is a 25:1 extract.

19. The dietary composition of any one of claims 1-18, wherein:
the bean extract is a Phaseolus vulgaris extract;
the dairy protein is a combination of whey protein isolate, whey protein concentrate, and calcium caseinate;
the fiber is a combination of inulin, beta-glucan, and soluble corn fiber;
the chromium is present as a chromium oligofructose complex.

20. The dietary composition of any one of claims 1-19, wherein the dietary composition is incorporated as part of a powdered shake mix.

21. The dietary composition of any one of claims 1-20, wherein the dietary composition stabilizes blood glucose concentration in a mammal.

22. The dietary composition of any one of claims 1-21, wherein the dietary composition slows the rate of glucose absorption in a mammal.

23. The dietary composition of any one of claims 1-22, wherein the dietary composition increases satiety in a mammal.

24. The dietary composition of any one of claims 1-23, wherein the dietary composition decreases hunger cravings in a mammal.
